(19) United States
(12) Patent Application Publication
(10) Pub. No.: US 2023/0168232 A1
(43) Pub. Date: Jun. 1, 2023

(54) SYSTEM AND METHOD FOR MONITORING EMISSION OF GREENHOUSE GAS

(71) Applicant: PING AN TECHNOLOGY (SHENZHEN) CO., LTD., Shenzhen (CN)

(72) Inventors: ANDY JIAYING WONG, South San Francisco, CA (US); HANG ZHOU, Santa Clara, CA (US); JUI-HSIN LAI, Santa Clara, CA (US); MEI HAN, Palo Alto, CA (US)

(73) Assignee: PING AN TECHNOLOGY (SHENZHEN) CO., LTD., Shenzhen (CN)

(21) Appl. No.: 17/540,205

(22) Filed: Dec. 1, 2021

(51) Int. Cl. G01N 33/00; G06F 30/27

(52) U.S. Cl. CPC G01N 33/0075; G06F 30/27

(57) ABSTRACT

A system and a method for monitoring emission of a greenhouse gas are disclosed. A plurality of satellite observations associated with the emission of the greenhouse gas in a first region of interest are received from a plurality of satellite data sources, respectively. The plurality of satellite observations are fused to generate a fused input data set. An emission estimation model is used to generate a first emission estimate of the greenhouse gas in the first region of interest based on the fused input data set.

### TECHNICAL FIELD

The present disclosure relates to gas emission monitoring, and more particularly, to a system and method for monitoring emission of a greenhouse gas in a region of interest.

### BACKGROUND

Quantification of anthropogenic carbon dioxide (CO₂) emission at point sources (also referred to as emission sources herein) and governance scales are fundamental to implement, monitor and evaluate progress toward carbon neutrality. For example, implementing cap-and-trade programs and carbon tax requires verification of CO₂ emission at respective power plants and factories. In another example, at a governance scale, understanding the heterogeneity of CO₂ emission across cities and counties are helpful for policy implementation to effectively reduce emission of CO₂. In yet another example, at an international level, disputes on national inventories of greenhouse gas emission hinder the implementation of carbon border adjustment tax and the attribution of national responsibility for climate changes. These disputes may be solved with an aid of a quantification of the CO₂ emission.

### SUMMARY

In one aspect, a method for monitoring emission of a greenhouse gas is disclosed. A plurality of satellite observations associated with the emission of the greenhouse gas in a first region of interest are received from a plurality of satellite data sources, respectively. The plurality of satellite observations are fused to generate a fused input data set. An emission estimation model is used to generate a first emission estimate of the greenhouse gas in the first region of interest based on the fused input data set.

In another aspect, a system for monitoring emission of a greenhouse gas is disclosed. The system includes a memory and a processor. The memory is configured to store instructions. The processor is coupled to the memory and configured to execute the instructions to perform a process including: receiving a plurality of satellite observations associated with the emission of the greenhouse gas in a first region of interest from a plurality of satellite data sources, respectively; fusing the plurality of satellite observations to generate a fused input data set; and using an emission estimation model to generate a first emission estimate of the greenhouse gas in the first region of interest based on the fused input data set.

In yet another aspect, a non-transitory computer-readable storage medium is disclosed. The computer-readable storage medium is configured to store instructions which, in response to an execution by a processor, cause the processor to perform a process including: receiving a plurality of satellite observations associated with the emission of the greenhouse gas in a first region of interest from a plurality of satellite data sources, respectively; fusing the plurality of satellite observations to generate a fused input data set; and using an emission estimation model to generate a first emission estimate of the greenhouse gas in the first region of interest based on the fused input data set.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated herein and form a part of the specification, illustrate implementations of the present disclosure and, together with the description, further serve to explain the present disclosure and to enable a person skilled in the pertinent art to make and use the present disclosure.

FIGS. 1A-1C are graphical representations illustrating an exemplary implementation of a Gaussian plume model for estimating CO₂ emission, according to some examples.

FIG. 2 illustrates a block diagram of an exemplary operating environment for a system configured to monitor emission of a greenhouse gas, according to embodiments of the disclosure.

FIG. 3A illustrates an exemplary process for monitoring emission of a greenhouse gas, according to embodiments of the disclosure.

FIG. 3B illustrates another exemplary process for monitoring emission of a greenhouse gas, according to embodiments of the disclosure.

FIG. 3C illustrates yet another exemplary process for monitoring emission of a greenhouse gas, according to embodiments of the disclosure.

FIG. 4 illustrates an exemplary disaggregation process for disaggregating a greenhouse-gas emission value associated with a grid to one or more disaggregated greenhouse-gas emission values for one or more emission sources within the grid, according to embodiments of the disclosure.

FIG. 5 is a flowchart of an exemplary method for monitoring emission of a greenhouse gas, according to embodiments of the disclosure.

FIG. 6 is a flowchart of another exemplary method for monitoring emission of a greenhouse gas, according to embodiments of the disclosure.

Implementations of the present disclosure will be described with reference to the accompanying drawings.

### DETAILED DESCRIPTION

Reference will now be made in detail to the exemplary embodiments, examples of which are illustrated in the accompanying drawings. Wherever possible, the same reference numbers will be used throughout the drawings to refer to the same or like parts.

A traditional approach to quantify greenhouse gas emission, such as CO₂ emission, includes multiplying an emission-generating activity (e.g., a cement production activity) with an emission factor that specifies the greenhouse gas emission per unit of activity. While this approach is simple in concept, it may experience various challenges in execution. For example, the emission factor is not only region specific, but also is differentiated by technology and operating conditions. A derivation of the emission factor may not be feasible in many developing countries and at a nationwide local-scale level. In another example, a report for the emission-generating activity provided by an emission source itself can be subject to delays, errors, and frauds. In yet another example, the accuracy and consistency of an emission estimate of the greenhouse gas can be greatly affected by a data source and model assumption.

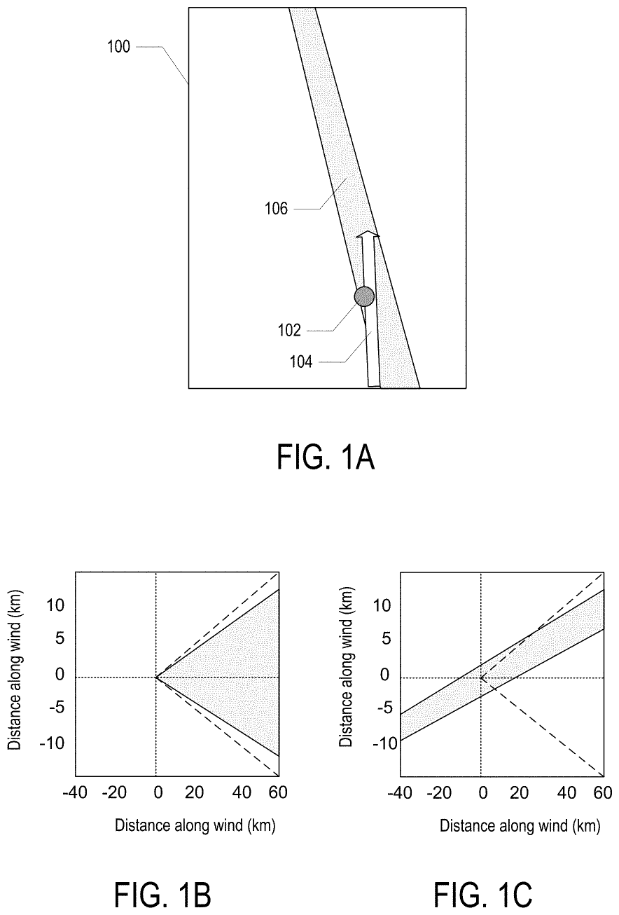

An alternative approach to monitor CO₂ emission uses satellite observations of column-averaged dry-air mole fractions of CO₂ in the atmosphere (xCO₂), which may be referred to as a satellite remote-sensing based approach. For example, a Gaussian plume model can be applied to estimate CO₂ emission at a scale of a point source using the xCO₂ satellite observations. The Gaussian plume model is described below in more detail with reference to FIGS. 1A-1C below. It has been demonstrated that a Gaussian plume can be fitted over xCO₂ data obtained from the Orbiting Carbon Observatory 2 (OCO-2) satellite. By associating the plume with its nearby emission source (e.g., a power plant) and by quantifying the difference in xCO₂ between the background and the plume, daily averaged CO₂ emission of the emission source can be estimated when xCO₂ data from the OCO-2 satellite is available.

The satellite remote-sensing based approach can be promising because it can address issues of the traditional approach. For example, xCO₂ data from the OCO-2 satellite covers the globe with a spatial resolution of 2.25 km per pixel. Besides, xCO₂ data from the greenhouse gases observing satellite 1 (GOSAT-1) and GOSAT-2 may have a spatial resolution of 10 km per pixel. Therefore, estimation of CO₂ emission can be potentially implemented at a local scale globally. In another example, the xCO₂ data from the OCO-2 satellite are available to the public within days of an initial observation, and gridded spatial estimates of CO₂ emission may be validated with ground measurement sensors (e.g., a gas analyzer). Thus, this approach can be less susceptible to fraud and delay, and different estimation methods can be systematically evaluated with the same set of field measurements.

However, the satellite remote-sensing based approach may suffer from several technical issues and bottlenecks. For example, the OCO-2 satellite may fail to provide sufficient xCO₂ data for a desired temporal and spatial coverage. Specifically, the OCO-2 satellite may provide measurement of xCO₂ with a narrow ground field of view (eight 2.25-by-2.25 km² pixels) in perpendicular to satellite overpass. Although the OCO-2 satellite can cover the globe every 16 days, the narrow field of view may cause large spatial gaps between swaths. Furthermore, the presence of clouds and smog may make the xCO₂ data collected by the OCO-2 satellite invalid. With the Gaussian plume model having specific image criteria for estimating emission, it has been reported that only one image from a two-year period was suitable at a particular point source of interest.

In another example, the Gaussian plume model in the satellite remote-sensing based approach is applicable to isolated middle and large size point sources, which is not feasible for estimating CO₂ emission for individual point sources that are clustered in a small region. In yet another example, previous studies in the satellite remote-sensing based approach have only demonstrated the feasibility of estimating CO₂ emission using this approach at limited locations with manually selected data. It can be labor intensive to implement this approach at a daily and global scale.

To address one or more of the aforementioned issues, the present disclosure provides a system and method for monitoring emission of a greenhouse gas by leveraging satellite data fusion and proxy estimation. The greenhouse gas described herein may be any gas that can trap heat in the atmosphere and warm the planet and may include CO₂, methane, nitrous oxide, fluorinated gases, etc. Without loss of generality, CO₂ may be used as an example of the greenhouse gas in the following description of the present disclosure.

In the system and method disclosed herein, satellite observations associated with the emission of the greenhouse gas (e.g., including various xCO₂ data from OCO-2, OCO-3, and other satellites) can be fused to generate a fused xCO₂ input data set to enable a better approximation of an extent of a plume as well as xCO₂ values of both the background and the plume. Daily observations of wind and other industrial gases (e.g., methane) may be used to guide a reconstruction of the plume outside the field of view of the OCO-2 or OCO-3 satellite. A gap-filling technique can be used to increase the number of satellite images that may be suitable for the Gaussian plume model. Besides, observations of other reference resources (e.g., methane, NO₂, or heat signals) can be used as a proxy to estimate CO₂ emission when xCO₂ data is not available. Additionally, a disaggregation process can be applied to attribute a satellite-based emission estimate having a coarse resolution into a fine-scale emission map by facility objects (e.g., a cluster of factories).

The system and method disclosed herein can improve the applicability of the Gaussian plume model for monitoring emission of the greenhouse gas from point sources with a short delay. For example, emission estimates can be available within 2-3 days after the emission of the greenhouse gas, which is well suited for monitoring and enforcing the carbon neutral policy. The system and method disclosed herein can significantly increase the frequency of satellite-based emission estimates by using a proxy estimation approach and can enable emission estimation for clusters of small factories using a disaggregation process. Implementation of the proxy estimation approach over a large area may allow estimation of industrial emission at governance scales.

FIGS. 1A-1C are graphical representations illustrating an exemplary implementation of a Gaussian plume model for estimating CO₂ emission, according to some examples. Referring to FIG. 1A, an image 100 shows a wind direction 104 and a point source 102 that emits CO₂. Point source 102 can be, for example, a power plant, a cement production factory, a steelmaking plant, or any other plant or factory that emits a greenhouse gas, such as CO₂. A stripe 106 shows an OCO-2 overpass or flyby, correlating xCO₂ data from the OCO-2 satellite to part of the CO₂ emission from point source 102. A Gaussian plume model can be used to fit and model the xCO₂ data from the OCO-2 satellite to provide an estimate of CO₂ emission associated with point source 102.

For example, for each overpass or flyby, the magnitude of the vector mean of the ERA-Interim and MERRA2 winds can be taken as a wind speed to model a plume. The Gaussian plume model equations can be expressed in the following expressions (1) and (2):

$$V ( x , y ) = \frac{F}{\sqrt{2 π} {σ}_{y} ( x ) u} {e}^{- \frac{1}{2} {( \frac{y}{{σ}_{y} ( x )} )}^{2}} \qquad ( 1 )$$

$${σ}_{y} ( x ) = a \cdot {( \frac{x}{{x}_{0}} )}^{0.894} . \qquad ( 2 )$$

In the above expressions (1) and (2), V denotes the CO₂ vertical column in g/m² at and downwind of a point source. The x direction is parallel to the wind direction, and x denotes a distance (in meter (m)) parallel to the wind direction from the point source. The y direction is perpendicular to the wind direction, and y denotes a distance (in m) across the wind direction. V depends on an emission rate F (in g/s), the across wind distance y (in m), a wind speed u (in m/s), and a standard deviation in the y direction (e.g., σ_{y} (in m)). x₀=1000 m can be a characteristic length so that the argument of the exponent is dimensionless. a denotes the atmospheric stability parameter, which can be determined by classifying a source environment by the Pasquill-Gifford stability, which depends on the surface wind speed, cloud cover, and time of day. The surface wind speed and cloud cover can be taken from ERA-Interim.

A region of the OCO-2 swath (e.g., upwind and thus not affected by the point source) can be selected as the background, and the xCO₂ from these points in the region can be averaged. The model plume can then be defined as an area from the x axis (wind vector) down to a predetermined threshold of intensity (e.g., a threshold of 5% intensity) in the positive and negative y directions. The observed plume can be defined based on the points that correspond to the model plume, accounting for the light path. An extent of the plume can be defined by V(x,y) in the above expressions (1) and (2), with V(x,y) being greater than the predetermined threshold of intensity (e.g., 5% greater than the background concentration of CO₂ or another emission gas). For example, the extent of plume can be an area defined by V(x,y) in which V(x,y) is greater than the predetermined threshold of intensity (e.g., 5% greater than the background concentration of CO₂).

FIG. 1B shows an exemplary xCO2 plume relative to the background. FIG. 1C shows an exemplary xCO2 plume relative to the background as would be viewed by the OCO-2 satellite. Dashed lines in FIGS. 1B-1C show a 5% plume density cutoff from the axial value of the Gaussian plume model.

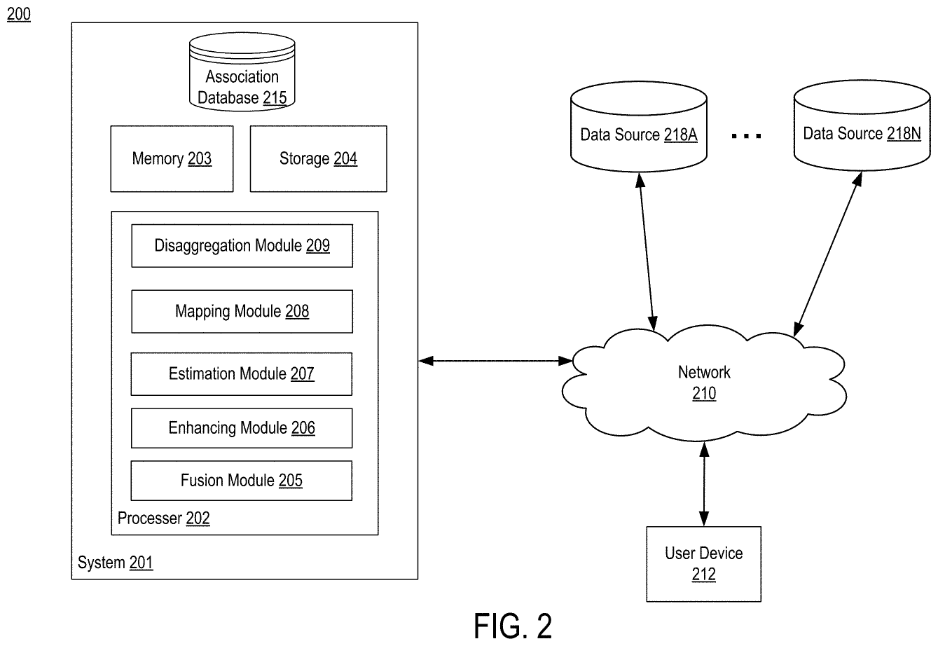

Given limitations of data availability from one or more satellites, emission estimation of the greenhouse gas (e.g., CO₂) across a large region at a point source scale and/or a governance scale can be challenging. To address this challenge, a system and method for monitoring emission of the greenhouse gas are disclosed herein, which are described below in more detail with reference to FIGS. 2-6. In the system and method disclosed herein, availability of input data to an emission estimation model (e.g., the Gaussian plume model) can be increased by compiling and fusing xCO₂ observations from multiple satellites. Also, the quantity of suitable input data to the emission estimation model can be increased by enhancing xCO₂ data with concurrent industrial gas data. Furthermore, by deriving and applying a mapping relationship between CO₂ emission and one or more reference sources, CO₂ emission estimates can be gap-filled in regions where no xCO₂ data is available. The one or more reference sources may include an industrial gas emission such as methane, land surface temperature, or shortwave infrared heat signals. Additionally, a spatial resolution of CO₂ emission estimates can be further improved by disaggregating a total emission value over a grid to a plurality of identifiable emission sources within the grid. Thus, the system and method disclosed herein can provide rigorous, consistent, and efficient CO₂ emission estimates for different emission sources around the world.

FIG. 2 illustrates a block diagram of an exemplary operating environment 200 for a system 201 configured to monitor emission of a greenhouse gas, according to embodiments of the disclosure. Operating environment 200 may include system 201, a user device 212, and a plurality of data sources 218A, . . . , 218N. Components of operating environment 200 may be coupled to each other through a network 210.

In some embodiments, system 201 may be embodied on a cloud computing device. Alternatively, system 201 may be embodied on a local computing device. The computing device can be, for example, a server, a desktop computer, a laptop computer, a tablet computer, or any other suitable electronic device including a processor and a memory. In some embodiments, system 201 may include a processor 202, a memory 203, a storage 204, and an association database 215. It is understood that system 201 may also include any other suitable components for performing functions described herein.

For example, system 201 may have different components in a single device, such as an integrated circuit (IC) chip, or separate devices with dedicated functions. The IC may be implemented as an application-specific integrated circuit (ASIC) or a field-programmable gate array (FPGA). In another example, one or more components of system 201 may be located in a cloud computing environment, or may be alternatively in a single location or distributed locations but communicate with each other through network 210.

Processor 202 may include any appropriate type of general-purpose or special-purpose microprocessor, digital signal processor, microcontroller, graphics processing unit (GPU), etc. Processor 202 may include one or more hardware units (e.g., portion(s) of an integrated circuit) designed for use with other components or to execute part of a program. The program may be stored on a computer-readable medium, and when executed by processor 202, it may perform one or more functions. Processor 202 may be configured as a separate processor module dedicated to monitoring emission of the greenhouse gas. Alternatively, processor 202 may be configured as a shared processor module for performing other functions.

Processor 202 may include several modules, such as a fusion module 205, an enhancing module 206, an estimation module 207, a mapping module 208, and a disaggregation module 209. Although FIG. 2 shows that fusion module 205, enhancing module 206, estimation module 207, mapping module 208, and disaggregation module 209 are within one processor 202, they may also be likely implemented on different processors located closely or remotely with each other. For example, mapping module 208 may be implemented by a processor (e.g., a GPU) dedicated to off-line training of a machine learning model for deriving a mapping relationship, and estimation module 207 may be implemented by another processor for estimating emission of the greenhouse gas based on the mapping relationship.

Fusion module 205, enhancing module 206, estimation module 207, mapping module 208, and disaggregation module 209 (and any corresponding sub-modules or sub-units) can be hardware units (e.g., portions of an integrated circuit) of processor 202 designed for use with other components or software units implemented by processor 202 through executing at least part of a program. The program may be stored on a computer-readable medium, such as memory 203 or storage 204, and when executed by processor 202, it may perform one or more functions.

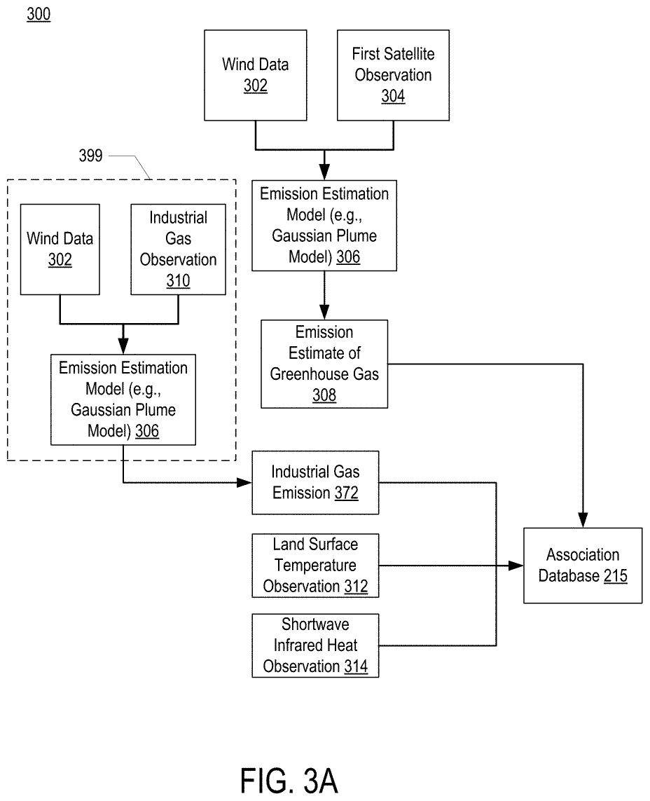

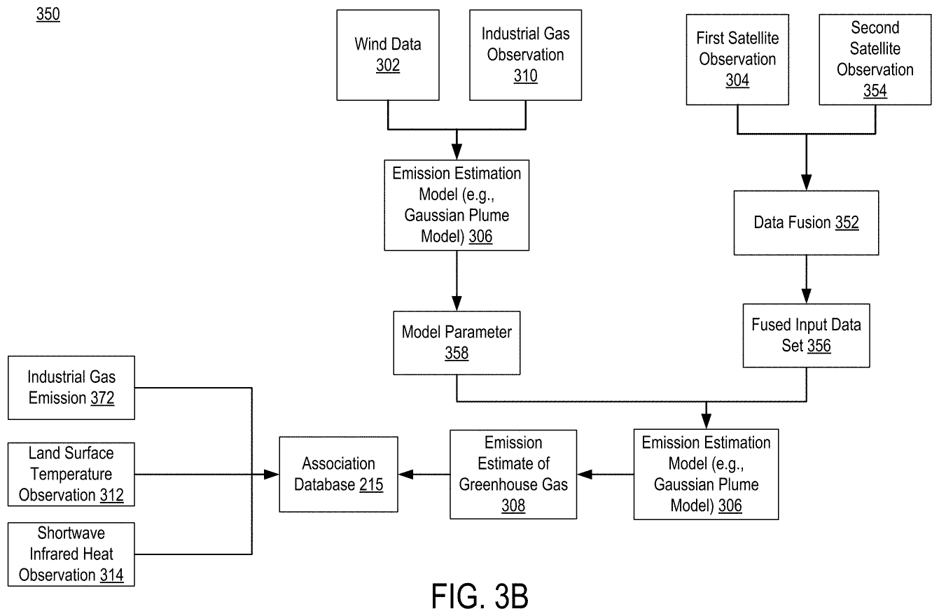

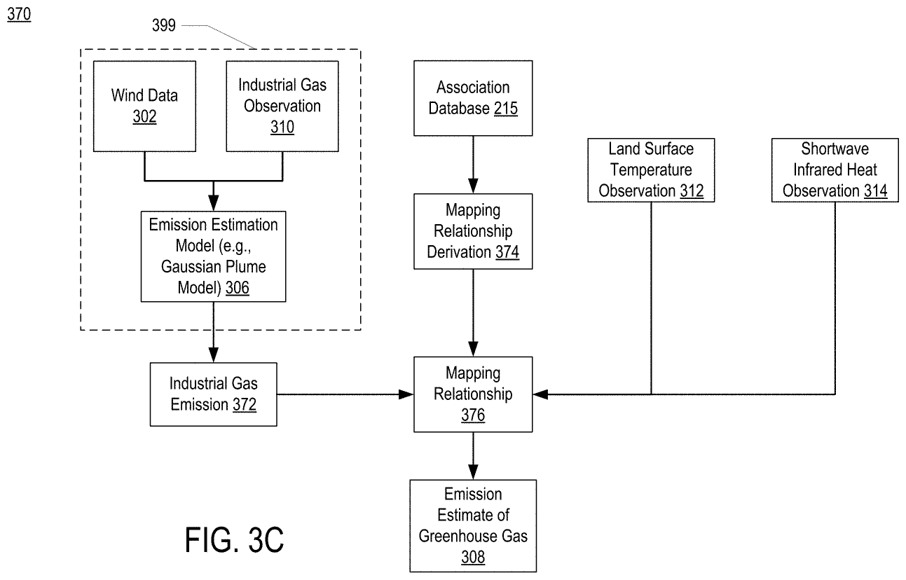

Fusion module 205, enhancing module 206, estimation module 207, mapping module 208, disaggregation module 209, and association database 215 are described below in more detail with reference to FIGS. 3A-6.

Memory 203 and storage 204 may include any appropriate type of mass storage provided to store any type of information that processor 202 may need to operate. For example, memory 203 and storage 204 may be a volatile or non-volatile, magnetic, semiconductor-based, tape-based, optical, removable, non-removable, or other type of storage device or tangible (i.e., non-transitory) computer-readable medium including, but not limited to, a ROM, a flash memory, a dynamic RAM, and a static RAM. Memory 203 and/or storage 204 may be configured to store one or more computer programs that may be executed by processor 202 to perform functions disclosed herein. For example, memory 203 and/or storage 204 may be configured to store program(s) that may be executed by processor 202 to estimate emission of the greenhouse gas. Memory 203 and/or storage 204 may be further configured to store information and data used by processor 202.

User device 212 can be a computing device including a processor and a memory. For example, user device 212 can be a desktop computer, a laptop computer, a tablet computer, a smartphone, a game controller, a television (TV) set, a music player, a wearable electronic device such as a smart watch, an Internet-of-Things (IoT) appliance, a smart vehicle, or any other suitable electronic device with a processor and a memory. User device 212 may be operated by a user. In some embodiments, user device 212 may receive a request for estimating emission of the greenhouse gas in a region of interest from a user and may forward the request to system 201, causing system 201 to generate an emission estimate of the greenhouse gas in the region of interest. System 201 may send the emission estimate of the greenhouse gas to user device 212, so that user device 212 may present the emission estimate of the greenhouse gas to the user via a screen of user device 212.

The plurality of data sources 218A, . . . , 218N may include a plurality of satellite data sources configured to store a plurality of satellite observations associated with the emission of the greenhouse gas in a region of interest, respectively. For example, the plurality of satellite data sources may include one or more of the following: an OCO-2 data source configured to store xCO₂ data from the OCO-2 satellite, an OCO-3 data source configured to store xCO₂ data from the OCO-3 satellite, a GOSAT-1 data source configured to store xCO₂ data from the GOSAT-1 satellite, a GOSAT-2 data source configured to store xCO₂ data from the GOSAT-2 satellite, or a TANSAT data source configured to store xCO₂ data from the TANSAT satellite.

The plurality of data sources 218A, . . . , 218N may also include a plurality of reference data sources configured to store observations of a plurality of reference resources in a region of interest, respectively. For example, the plurality of reference resources may include at least one of an industrial gas resource, a land surface temperature resource, or a shortwave infrared heat resource. The plurality of reference data sources may include one or more of the following: an industrial-gas data source configured to store industrial gas observations obtained from the sentinel-5P satellite, a land-surface-temperature data source configured to store land surface temperature observations obtained from the Landsat satellite, or a shortwave-infrared data source configured to store shortwave infrared heat observations obtained from Sentinel-2 infrared bands (e.g., for structures hotter than 1300K, such as a blast furnace).

FIG. 3A illustrates an exemplary process 300 for monitoring emission of a greenhouse gas in a region of interest, according to embodiments of the disclosure. In process 300, the region of interest may include one or more point sources (e.g., power plants) that may emit the greenhouse gas. One or more first satellite observations 304 associated with the emission of the greenhouse gas in the region of interest can be obtained from one or more first satellite data sources. For example, the greenhouse gas may be CO₂. The one or more first satellite observations may include one or more first observations of column-averaged dry-air mole fractions of CO₂ in the atmosphere (xCO₂) (referred to as “xCO₂ observation” herein). The one or more first xCO₂ observations may include, for example, xCO₂ data from the OCO-2 satellite, xCO₂ data from the OCO-3 satellite, or both. In some cases, the one or more first xCO₂ observations may include sufficient xCO₂ data so that the CO₂ emission in the region of interest can be estimated using an emission estimation model 306 only based on the one or more first xCO₂ observations.

Initially, estimation module 207 may be configured to receive wind data 302 (such as a wind direction and a wind velocity) and one or more first satellite observations 304 (e.g., the one or more first xCO₂ observations). Estimation module 207 may input wind data 302 and one or more first satellite observations 304 into emission estimation model 306. Emission estimation model 306 can include, for example, a Gaussian plume model. Estimation module 207 may apply emission estimation model 306 to generate an emission estimate 308 of the greenhouse gas in the region of interest based on wind data 302 and one or more first satellite observations 304.

Mapping module 208 may associate emission estimate 308 of the greenhouse gas with one or more observations of one or more reference resources in the region of interest to generate a training data set. Mapping module 208 may store the training data set in association database 215. In some embodiments, the one or more observations of the one or more reference resources may include at least one of the following that is associated with the region of interest: an industrial gas observation 310, a land surface temperature observation 312, or a shortwave infrared heat observation 314. An industrial gas emission 372 can be estimated using emission estimation model 306 based on wind data 302 and industrial gas observation 310. Mapping module 208 may associate emission estimate 308 of the greenhouse gas with each of industrial gas observation 310 (or equivalently, industrial gas emission 372), land surface temperature observation 312, and shortwave infrared heat observation 314. For example, mapping module 208 may pair emission estimate 308 of the greenhouse gas with each of industrial gas observation 310 (or equivalently, industrial gas emission 372), land surface temperature observation 312, and shortwave infrared heat observation 314 to generate the training data set. The training data set is described below in more detail with reference to FIG. 3C.

FIG. 3B illustrates another exemplary process 350 for monitoring emission of a greenhouse gas in a region of interest, according to embodiments of the disclosure. In process 350, the region of interest may include one or more point sources that may emit the greenhouse gas. One or more first satellite observations 304 associated with the emission of the greenhouse gas in the region of interest can be obtained from one or more first satellite data sources. One or more second satellite observations 354 associated with the emission of the greenhouse gas in the region of interest can be obtained from one or more second satellite data sources.

For example, the greenhouse gas may be CO₂. One or more first satellite observations 304 may include one or more first xCO₂ observations including, for example, xCO₂ data from the OCO-2 satellite, xCO₂ data from the OCO-3 satellite, or both. One or more second satellite observations 354 may include one or more second xCO₂ observations including, for example, at least one of the following: xCO₂ data from the GOSAT-1 satellite, xCO₂ data from the GOSAT-2 satellite, or xCO₂ data from the TANSAT satellite. The xCO₂ data from the various satellites may vary by precision (0.5-4 ppm), a spatial resolution (2-10 km), a field of view, or an orbit revisit time (3-16 days). The xCO₂ data from the OCO-2 or OCO-3 satellite may have a higher spatial resolution than that from the GOSAT-1, GOSAT-2 or TANSAT satellite.

In some embodiments, the one or more first xCO₂ observations may include sufficient xCO₂ data for Gaussian plume fitting so that the CO₂ emission in the region of interest can be estimated only based on the one or more first xCO₂ observations. Alternatively, the one or more first xCO₂ observations may not include sufficient xCO₂ data for Gaussian plume fitting so that the one or more first xCO₂ observations may be fused with the one or more second xCO₂ observations to estimate the CO₂ emission in the region of interest.

Fusion module 205 may be configured to perform an operation of data fusion 352 based on a plurality of satellite observations to generate a fused input data set 356 for the region of interest. For example, fusion module 205 may fuse one or more first satellite observations 304 with one or more second satellite observations 354 to generate fused input data set 356. The region of interest may include a geographical region that is divided into a plurality of grids (e.g., a plurality of 2.25-by-2.25 km² pixels). Through the operation of data fusion 352, a spatial coverage of xCO₂ data as an input to emission estimation model 306 can be improved. An output grid of the operation of data fusion 352 may have an xCO₂ data spatial resolution of about 2 km (e.g., 2 km, ±5% of 2 km, ±10% of 2 km, etc.), with a box boundary encompassing the region of interest (e.g., including both the emission point source and a CO₂ plume from the Gaussian plume model).

Specifically, fusion module 205 may resample each satellite observation into one or more resampled observation values associated with one or more grids in the plurality of grids. For example, each satellite observation may include one or more initial observation values (e.g., initial xCO₂ values) associated with the region of interest obtained from a corresponding satellite data source. Fusion module 205 may apply a geographically-weighted method to generate the one or more resampled observation values (e.g., resampled xCO₂ values) associated with the one or more grids based on the one or more initial observation values. Exemplary geographically-weighted methods may include, but not limited to, an average of neighbors, a nearest Gaussian weighting method, or any other suitable weighting method. Next, for each grid in the plurality of grids, fusion module 205 may determine an availability of resampled observation values associated with the grid. Fusion module 205 may generate a fused input value associated with the grid based on the availability of resampled observation values associated with the grid. Then, fusion module 205 may generate fused input data set 356 to include a corresponding fused input value for each of the plurality of grids.

For example, one or more first initial observation values associated with the region of interest can be extracted from the xCO₂ data obtained from the OCO-2 satellite and/or the OCO-3 satellite, and can be resampled onto the plurality of grids using a geographically-weighted method to generate one or more first resampled observation values associated with one or more first grids. The one or more first grids may be at least part of the plurality of grids, and the remaining grids in the plurality of grids may have no first resampled observation values. Similarly, one or more second initial observation values associated with the region of interest can be extracted from the xCO₂ data from the GOSAT-1, GOSAT-2 and/or TANSAT satellite, and can be resampled onto the plurality of grids using a geographically-weighted method to generate one or more second resampled observation values associated with one or more second grids. The one or more second grids may be at least part of the plurality of grids, and the remaining grids in the plurality of grids may have no second resampled observation values. The one or more first grids may or may not overlap with the one or more second grids.

With respect to a grid having both a first resampled observation value and a second resampled observation value (that is, the grid is an overlapped grid between the one or more first grids and the one or more second grids), fusion module 205 may determine a fused input value (e.g., a fused xCO₂ input value) associated with the grid to be an average of the first resampled observation value and the second resampled observation value. Then, an average resampled observation value in an overlapped area between the one or more first grids and the one or more second grids (denoted as xCO_{2(oco)}) can be calculated as an average of fused input values of overlapped grids between the one or more first grids and the one or more second grids.

With respect to a grid having either a first resampled observation value or a second resampled observation value (that is, the grid is only in the one or more first grids or the one or more second grids, but not both), fusion module 205 may determine a fused input value associated with the grid to be either the first resampled observation value or the second resampled observation value.

With respect to a grid that has no resampled observation value (that is, the grid is not included in the one or more first grids and the one or more second grids), fusion module 205 may compute a fused input value for the grid using the following expression (3):

xCO_{2(filled)}=((n_{coarse}−n_{oco})×(xCO_{2(coarse)})−xCO_{2(oco)})/n_{coarse} (3)

In the above expression (3), xCO_{2(filled)} denotes the fused input value (e.g., a fused xCO₂ input value) associated with the grid, n_{coarse} denotes a total number of grids (e.g., 2.25-by-2.25 km² pixels) associated with each single satellite observation, xCO_{2(coarse)} denotes an initial observation value (e.g., an initial xCO₂ value) obtained from the GOSAT-1, GOSAT-2 or TANSAT satellite observation, and n_{oco} denotes a total number of overlapped grids (e.g., overlapped 2.25-by-2.25 km² pixels) between the one or more first grids and the one or more second grids.

Enhancing module 206 may be configured to enhance fused input data set 356 using an industrial gas observation 310 associated with the region of interest. Specifically, enhancing module 206 may apply wind data 302 and industrial gas observation 310 to emission estimation model 306 to estimate one or more model parameters 358 associated with emission estimation model 306. Emission estimation model 306 may include a Gaussian plume model, and one or more model parameters 358 may include an extent of a plume in the Gaussian plume model.

In some embodiments, the atmospheric concentration of some industries can be observed by satellite, and there are many approaches to estimate their ground emission. Then, daily industrial gas products from the Sentinel-5P satellite (e.g., methane, carbon monoxide, sulfur dioxide, etc.) can be used to approximate an extent of a CO₂ plume in the Gaussian plume model. For example, if a CO₂-emitting point source concurrently emits sulfur dioxide, a Gaussian plume can be fitted over the sulfur dioxide data to determine an emission rate and an extent of the plume. Next, the emission rate and the extent of the plume determined from the sulfur dioxide data can be used to estimate a CO₂ emission. For illustration purposes, it is assumed that the chemical reaction is negligible and that the travel and dispersion rate are similar among emitted gases. This assumption allows the extent of the plume determined from the sulfur dioxide data to be used directly as the extent of the plume for CO₂ emission estimation. If the estimated plume partially overlaps with actual OCO observations, the Gaussian plume model equation (e.g., the above expressions (1)-(2)) and the actual OCO observations can be used to back-calculate one or more parameters of the equation (e.g., including a CO₂ emission rate). In practice, a more complex computation involving chemical transport models may be used to convert the extent of the plume associated with one emission gas to the extent of the plume associated with another emission gas.

For example, when fitting the Gaussian plume model on a non-CO₂ air pollution emission gas (e.g., methane), the wind direction may be tuned so that V (the vertical column of pollution across a location (x,y)) may optimally agree with a shape of the pollution plume on a satellite image. Then, based on a wind speed u and the atmospheric stability parameter a, an emission rate of the emission gas such as methane (denoted as F_methane) can be estimated. The parameters such as the tuned wind direction, the emission rate of methane (F_methane), the wind speed u, and the atmospheric stability parameter a can be reused in the estimation of CO₂ emission. For example, by assuming that CO₂ emission is proportional to the air pollution emission gas (e.g., methane) with a ratio r, a CO₂ emission rate (denoted as F_CO₂) in the Gaussian plume model can be written as F_CO₂=r×F_methane, where r is to be optimized such that V(x,y) of the Gaussian plume model for CO₂ emission best fits the limit data availability of xCO₂ data from the OCO-2 satellite and/or OCO-3 satellite. Then, an extent of the plume from the Gaussian plume model can be determined based on one or more of the following: the emission rate of CO₂ (F_CO₂), the tuned wind direction, the wind speed u, and the atmospheric stability parameter a based on the above expressions (1) and (2).

Estimation module 207 may be configured to apply fused input data set 356 to emission estimation model 306 to generate emission estimate 308 of the greenhouse gas in the region of interest based on one or more model parameters 358. Mapping module 208 may be configured to associate emission estimate 308 of the greenhouse gas with each of industrial gas emission 372 (or equivalently, industrial gas observation 310), land surface temperature observation 312, and shortwave infrared heat observation 314 to generate a training data set. Mapping module 208 may store the training data set in association database 215.

FIG. 3C illustrates yet another exemplary process 370 for monitoring emission of a greenhouse gas, according to embodiments of the disclosure. Process 370 illustrates a proxy estimation approach to estimate an emission of the greenhouse gas when no satellite observation associated with the emission of the greenhouse gas is available in a region of interest. In the proxy estimation approach, a mapping relationship can be derived between the emission of the greenhouse gas and one or more reference resources for each grid in the region of interest. In some embodiments, the mapping relationship can be established by emission infrastructure category (e.g., coal power plants versus natural gas power plants) or by a search distance from a point source of interest. The derived mapping relationship can be applied to estimate CO₂ emission in a scenario where xCO₂ data is absent, or applied to identify abnormal estimates by the Gaussian plume model.

Specifically, mapping module 208 may be configured to perform an operation of mapping relationship derivation 374 to determine a mapping relationship 376 between the emission of the greenhouse gas and one or more reference resources based on association database 215. The one or more reference resources may include at least one of an industrial gas resource, a land surface temperature resource, or a shortwave infrared heat resource.

Next, estimation module 207 may be configured to obtain one or more observations of the one or more reference resources in the region of interest, respectively. Estimation module 207 may determine emission estimate 308 of the greenhouse gas in the region of interest based on mapping relationship 376 and the one or more observations of the one or more reference resources. The one or more observations of the one or more reference resources may include at least one of industrial gas observation 310, land surface temperature observation 312, or shortwave infrared heat observation 314. For example, an industrial gas emission 372 can be estimated using emission estimation model 306 based on wind data 302 and industrial gas observation 310. Estimation module 207 may apply mapping relationship 376 to generate emission estimate 308 of the greenhouse gas based on industrial gas emission 372, land surface temperature observation 312, and shortwave infrared heat observation 314.

In some embodiments, mapping relationship 376 can be modeled by a mapping model. The mapping model may be a Gaussian process regression model, a random forest model, a multivariate regression model, a gradient boosted decision tree mode, a neural network model, or any other suitable model. The mapping model may take at least one of industrial gas observation 310 (or industrial gas emission 372), land surface temperature observation 312, or shortwave infrared heat observation 314 as an input, and may generate emission estimation 308 of the greenhouse gas as an output. For example, mapping module 208 may be configured to train the mapping model using training data sets stored in association database 215. Estimation module 207 may apply the trained mapping model to generate emission estimate 308 of the greenhouse gas based on one or more of the following: industrial gas observation 310 (or industrial gas emission 372), land surface temperature observation 312, or shortwave infrared heat observation 314.

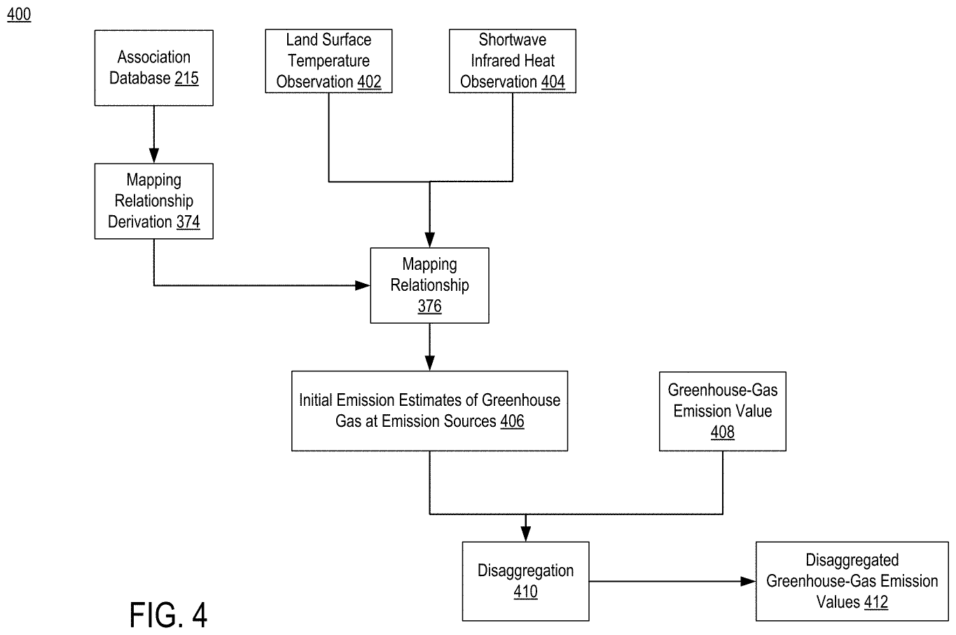

FIG. 4 illustrates an exemplary disaggregation process 400 for disaggregating a greenhouse-gas emission value associated with a grid to one or more disaggregated greenhouse-gas emission values for one or more emission sources within the grid, according to embodiments of the disclosure. In some embodiments, a region of interest may be divided into a plurality of grids, and an emission estimate of the greenhouse gas in the region of interest may include a plurality of greenhouse-gas emission values for the plurality of grids, respectively. For each grid in the plurality of grids, disaggregation module 209 may identify one or more emission sources within the grid, and may disaggregate a greenhouse-gas emission value 408 associated with the grid to one or more disaggregated greenhouse-gas emission values 412 for the one or more emission sources.

For example, mapping module 208 may perform an operation of mapping relationship derivation 374 to determine mapping relationship 376 based on association database 215. Mapping relationship 376 between the emission of the greenhouse gas and each of the land surface temperature and shortwave infrared heat signals can be used to disaggregate greenhouse-gas emission value 408 associated with the grid. To begin with, one or more emission sources within the grid may be classified and delineated from high-resolution satellite imageries, maps, and other ancillary data. If only an emission source of interest is present (e.g., a single potential emitter) at an upwind location from the plume, disaggregation module 209 may attribute greenhouse-gas emission value 408 of the grid to the emission source of interest, and no disaggregation of greenhouse-gas emission value 408 is needed. However, if multiple emission sources are identified as potential emitters (e.g., a cluster of factories), disaggregation module 209 may perform an operation of disaggregation 410 to disaggregate greenhouse-gas emission value 408.

Specifically, for each emission source from the multiple emission sources, disaggregation module 209 may obtain one or more observations of one or more reference resources associated with the emission source. The one or more observations of one or more reference resources may include at least one of the following: a land surface temperature observation 402 or a shortwave infrared heat observation 404. Land surface temperature observation 402 and shortwave infrared heat observation 404 may have spatial resolutions of about 100 m and 20 m, respectively, which are higher than a spatial resolution (e.g., 2 km) of greenhouse-gas emission value 408.

Next, disaggregation module 209 may use the one or more observations of the one or more reference resources (e.g., land surface temperature observation 402, shortwave infrared heat observation 404) to determine an initial emission estimate 406 of the greenhouse gas for the emission source based on mapping relationship 376. Thus, by performing similar operations, disaggregation module 209 may determine initial emission estimates 406 of the greenhouse gas at the multiple emission sources, respectively.

Then, disaggregation module 209 may perform an operation of disaggregation 410 to disaggregate greenhouse-gas emission value 408. For example, disaggregation module 209 may disaggregate greenhouse-gas emission value 408 to generate disaggregated greenhouse-gas emission values 412 for the identified emission sources based on initial emission estimates 406 of the greenhouse gas. Specifically, disaggregation module 209 may disaggregate greenhouse-gas emission value 408 to generate disaggregated greenhouse-gas emission values 412 using the following expressions (4) and (5):

CO₂₍₂ₖₘ₎═CO₂₍₁₎+ . . . +CO₂₍ₙ₎, (4)

CO₂₍ᵢ₎═(CO₂ₑ₍ᵢ₎/Σᵢ₌₁ⁿCO₂ₑ₍ᵢ₎)×CO₂₍₂ₖₘ₎, (5)

In the above expressions (4) and (5), n denotes the number of emission sources within the grid (e.g., the 2.25-by-2.25 km² pixel), CO₂₍₂ₖₘ₎ denotes greenhouse-gas emission value 408 at the grid, CO₂₍ᵢ₎ denotes a disaggregated greenhouse-gas emission value 412 at an emission source i, with 1≤i≤n, and CO₂ₑ₍ᵢ₎ denotes an initial emission estimate 406 at the emission source i. CO₂ₑ₍ᵢ₎ in the expression (5) may be determined based on mapping relationship 376 as described above.

In some embodiments, disaggregation module 209 may identify, from the one or more emission sources, an emission source having a potentially falsified emission report based on a disaggregated greenhouse-gas emission value associated with the emission source. Disaggregation module 209 may provide a notification to prioritize a field inspection on the emission source having the potentially falsified emission report.

Thus, disaggregation process 400 disclosed herein may be used to help government agencies to identify potential emission sources that have falsified emission reports. Through an application of disaggregation process 400, the government agencies may prioritize which region or which factory needs to be inspected by a field measurement team. For example, if a difference between a greenhouse-gas emission value 408 of a grid and a sum of reported emission values in the grid is less than a predefined error threshold, field trip inspection may not be needed in the grid. However, if the difference is equal to or greater than the predefined error threshold, an emission source with the largest difference between its disaggregated greenhouse-gas emission value 412 and reported emission value can be selected to prioritize for a field trip inspection. If the inspected or audited emission source does not commit fraud, its reported emission value can be verified and used to update the above expressions (4)-(5) and determine a next most suspicious emission source. Meanwhile, the reported emission value that is verified may also be used to improve mapping relationship 376. To further improve aggregation process 400 and expedite fraud identification, a ground measurement equipment may also be installed at an emission source whose disaggregated greenhouse-gas emission value 412 differs from its reported emission value significantly (e.g., a difference between the disaggregated greenhouse-gas emission value and the reported emission value is greater than an error threshold). As a result, measurements from the ground measurement equipment can be used to improve the monitoring of the emission of the greenhouse gas at the emission source.

With combined reference to FIGS. 2-4 described above, an exemplary operation process of monitoring emission of the greenhouse gas (e.g., CO₂) is provided herein. Initially, xCO₂ data from various satellite data sources (e.g., the OCO-2, OCO-3, GOSAT-1, GOSAT-2 and TANSAT satellites) associated with a region of interest are downloaded and fused to generate a fused xCO₂ input data set. The region of interest may be divided into a plurality of grids (e.g., each grid may have a size of 2 km×2 km). Concurrent gridded wind data (e.g., CFS v2), industrial gas data (e.g., Sentinel-5P atmospheric industrial gas data), land surface temperature data from the Landsat satellite (with a resolution of 100 m), and infrared heat data (e.g., Sentinel-2 longwave infrared data with a resolution of 20 m) can also be downloaded. The Gaussian plume model may be used to fit the fused xCO₂ input data set to generate a CO₂ emission estimate. Additionally or alternatively, the Gaussian plume model may be used to fit the industrial gas data to generate an emission estimate of the industrial gas.

In a case when the fused xCO₂ input data set cannot fulfill the data criteria of the Gaussian plume model, an xCO₂ enhancement procedure may be used to calculate one or more model parameters (e.g., including an extent of the xCO₂ Gaussian plume). Then, the Gaussian plume equations (1) and (2) may be used to back-calculate the CO₂ emission using the one or more model parameters.

If the estimation of the CO₂ emission in the region of interest is successful, the CO₂ emission estimate may be paired with concurrent observations of methane, land surface temperature and infrared heat data, and compiled into a training data set. The training data set may be used to develop a generalized or location-specific mapping relationship between the CO₂ emission and one or more of the industrial gas emission, the land surface temperature, and the shortwave infrared heat signals. The mapping relationship can be used to estimate the CO₂ emission at locations where xCO₂ data is unavailable.

In some cases, emission sources in a grid are clustered (e.g., closely located). The mapping relationship between the CO₂ emission and one or more of the land surface temperature and the shortwave infrared heat signals can also be used to disaggregate the CO₂ emission value associated with the grid into disaggregated CO₂ emission values for the individual emission sources.

Additionally, a mechanism that utilizes field-validated CO₂ emission values to improve the mapping relationship and the disaggregation approach can be implemented as described above with reference to FIG. 4. This mechanism can continuously improve the monitoring of the CO₂ emission, as government agencies dispatch auditing teams to inspect the CO₂ emission at the emission sources and more emission sensors are installed at the emission sources.

It is noted that in some application scenarios, industrial gas emission 372 may be disclosed and available to the public directly, and there is no need to derive industrial gas emission 372 from industrial gas observation 310 using emission estimation model 306 (as shown in a dashed-line box 399 of FIG. 3A or 3C). Thus, the derivation process shown in dashed-line box 399 of FIG. 3A or FIG. 3C may not be needed during the establishment of association database 215, the derivation of emission estimate 308 of the greenhouse gas, and/or the disaggregation 410 of greenhouse-gas emission value 408. That is, industrial gas emission 372 that is disclosed and available to the public can be used directly during the establishment of association database 215, the derivation of emission estimate 308 of the greenhouse gas, and/or the disaggregation 410 of greenhouse-gas emission value 408.

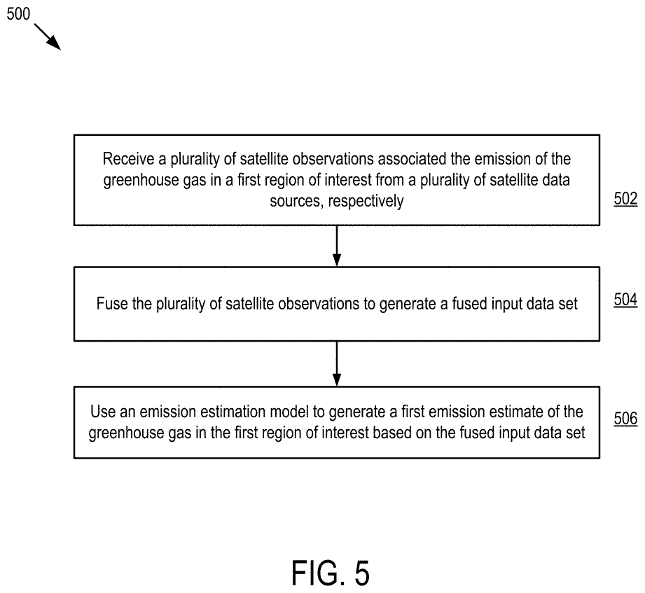

FIG. 5 is a flowchart of an exemplary method 500 for monitoring emission of a greenhouse gas, according to embodiments of the disclosure. Method 500 may be implemented by system 201, specifically fusion module 205 and estimation module 207, and may include steps 502-506 as described below. Some of the steps may be optional to perform the disclosure provided herein. Further, some of the steps may be performed simultaneously, or in a different order than those shown in FIG. 5.

At step 502, fusion module 205 may receive a plurality of satellite observations associated the emission of the greenhouse gas in a first region of interest from a plurality of satellite data sources, respectively.

At step 504, fusion module 205 may fuse the plurality of satellite observations to generate a fused input data set.

At step 506, estimation module 207 may use an emission estimation model to generate a first emission estimate of the greenhouse gas in the first region of interest based on the fused input data set.

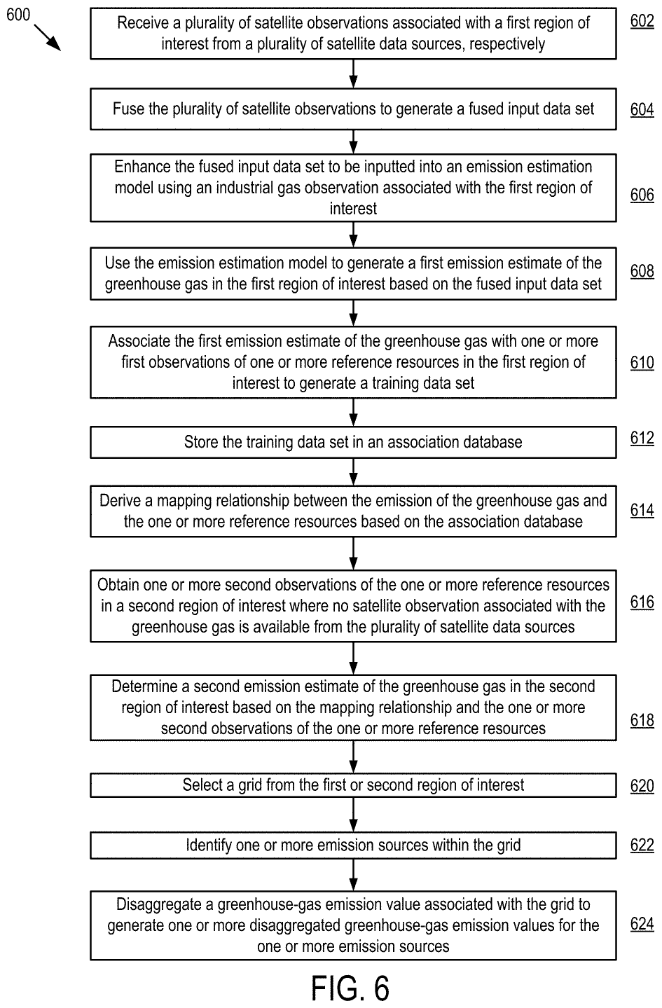

FIG. 6 is a flowchart of another exemplary method 600 for monitoring emission of a greenhouse gas, according to embodiments of the disclosure. Method 600 may be implemented by system 201, specifically fusion module 205, enhancing module 206, estimation module 207, mapping module 208, and disaggregation module 209, and may include steps 602-624 as described below. Some of the steps may be optional to perform the disclosure provided herein. Further, some of the steps may be performed simultaneously, or in a different order than those shown in FIG. 6.

At step 602, fusion module 205 may receive a plurality of satellite observations associated with a first region of interest from a plurality of satellite data sources, respectively.

At step 604, fusion module 205 may fuse the plurality of satellite observations to generate a fused input data set.

At step 606, enhancing module 206 may enhance the fused input data set to be inputted into an emission estimation model using an industrial gas observation associated with the first region of interest.

At step 608, estimation module 207 may use the emission estimation model to generate a first emission estimate of the greenhouse gas in the first region of interest based on the fused input data set. In some embodiments, the first region of interest may be divided into a plurality of grids. The first emission estimate of the greenhouse gas in the first region of interest may include a plurality of greenhouse-gas emission values for the plurality of grids, respectively.

At step 610, mapping module 208 may associate the first emission estimate of the greenhouse gas with one or more first observations of one or more reference resources in the first region of interest to generate a training data set.

At step 612, mapping module 208 may store the training data set in association database 215.

At step 614, mapping module 208 may derive a mapping relationship between the emission of the greenhouse gas and the one or more reference resources based on association database 215.

At step 616, estimation module 207 may obtain one or more second observations of the one or more reference resources in a second region of interest where no satellite observation associated with the greenhouse gas is available from the plurality of satellite data sources.

At step 618, estimation module 207 may determine a second emission estimate of the greenhouse gas in the second region of interest based on the mapping relationship and the one or more second observations of the one or more reference resources. In some embodiments, the second region of interest may be divided into a plurality of grids. The second emission estimate of the greenhouse gas in the second region of interest may include a plurality of greenhouse-gas emission values for the plurality of grids, respectively.

At step 620, disaggregation module 209 may select a grid from the first or second region of interest. A greenhouse-gas emission value associated with the grid can be obtained from the first emission estimate or the second emission estimate of the greenhouse gas.

At step 622, disaggregation module 209 may identify one or more emission sources within the grid.

At step 624, disaggregation module 209 may disaggregate the greenhouse-gas emission value associated with the grid to generate one or more disaggregated greenhouse-gas emission values for the one or more emission sources.

Another aspect of the disclosure is directed to a non-transitory computer-readable medium storing instructions which, when executed, cause one or more processors to perform the methods, as discussed above. The computer-readable medium may include volatile or non-volatile, magnetic, semiconductor, tape, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

According to one aspect of the present disclosure, a method for monitoring emission of a greenhouse gas is disclosed. A plurality of satellite observations associated with the emission of the greenhouse gas in a first region of interest are received from a plurality of satellite data sources, respectively. The plurality of satellite observations are fused to generate a fused input data set. An emission estimation model is used to generate a first emission estimate of the greenhouse gas in the first region of interest based on the fused input data set.

In some embodiments, the fused input data set to be inputted into the emission estimation model is enhanced using an industrial gas observation associated with the first region of interest.

In some embodiments, enhancing the fused input data set to be inputted into the emission estimation model using the industrial gas observation includes applying the industrial gas observation to the emission estimation model to estimate one or more model parameters associated with the emission estimation model.

In some embodiments, using the emission estimation model to generate the first emission estimate of the greenhouse gas in the first region of interest includes applying the fused input data set to the emission estimation model to generate the first emission estimate of the greenhouse gas based on the one or more model parameters.

In some embodiments, the emission estimation model includes a Gaussian plume model. The one or more model parameters include an extent of a plume in the Gaussian plume model.

In some embodiments, the first emission estimate of the greenhouse gas is associated with one or more first observations of one or more reference resources in the first region of interest to generate a training data set. The training data set is stored in an association database.

In some embodiments, the one or more reference resources include at least one of an industrial gas resource, a land surface temperature resource, or a shortwave infrared heat resource. The one or more first observations include at least one of the following that is associated with the first region of interest: an industrial gas observation, a land surface temperature observation, or a shortwave infrared heat observation.

In some embodiments, a mapping relationship between the emission of the greenhouse gas and the one or more reference resources is derived based on the association database.

In some embodiments, one or more second observations of the one or more reference resources are obtained in a second region of interest where no satellite observation associated with the emission of the greenhouse gas is available from the plurality of satellite data sources. A second emission estimate of the greenhouse gas in the second region of interest is determined based on the mapping relationship and the one or more second observations of the one or more reference resources.

In some embodiments, the first region of interest includes a geographical region that is divided into a plurality of grids. The first emission estimate of the greenhouse gas in the first region of interest includes a plurality of greenhouse-gas emission values for the plurality of grids, respectively.

In some embodiments, one or more emission sources are identified within a grid in the plurality of grids. A greenhouse-gas emission value associated with the grid is disaggregated to one or more disaggregated greenhouse-gas emission values for the one or more emission sources.

In some embodiments, disaggregating the greenhouse-gas emission value associated with the grid to the one or more disaggregated greenhouse-gas emission values for the one or more emission sources includes: for each emission source from the one or more emission sources, obtaining one or more observations of one or more reference resources associated with the grid, respectively; using the one or more observations of the one or more reference resources to determine an initial emission estimate of the greenhouse gas for the emission source based on a mapping relationship between the emission of the greenhouse gas and the one or more reference resources so that one or more initial emission estimates of the greenhouse gas are generated for the one or more emission sources; and disaggregating the greenhouse-gas emission value associated with the grid to the one or more disaggregated greenhouse-gas emission values based on the one or more initial emission estimates of the greenhouse gas for the one or more emission sources.

In some embodiments, an emission source having a potentially falsified emission report is identified from the one or more emission sources based on a disaggregated greenhouse-gas emission value associated with the emission source. A notification is provided to prioritize a field inspection on the emission source having the potentially falsified emission report.

In some embodiments, the first region of interest includes a geographical region that is divided into a plurality of grids. Fusing the plurality of satellite observations to generate the fused input data set includes: resampling each satellite observation into one or more resampled observation values associated with one or more grids in the plurality of grids; and for each grid in the plurality of grids, determining an availability of resampled observation values associated with the grid, and generating a fused input value associated with the grid based on the availability of resampled observation values associated with the grid.

In some embodiments, each satellite observation includes one or more initial observation values associated with the first region of interest. Resampling each satellite observation into the one or more resampled observation values associated with the one or more grids includes applying a geographically-weighted method to generate the one or more resampled observation values associated with the one or more grids based on the one or more initial observation values.

In some embodiments, the greenhouse gas includes CO₂. The plurality of satellite observations include a plurality of observations of column-averaged dry-air mole fractions of CO₂ in the atmosphere (xCO₂). The plurality of xCO₂ satellite observations are associated with the first region of interest and obtained from a plurality of xCO₂ data sources, respectively.

According to another aspect of the present disclosure, a system for monitoring emission of a greenhouse gas is disclosed. The system includes a memory and a processor. The memory is configured to store instructions. The processor is coupled to the memory and configured to execute the instructions to perform a process including: receiving a plurality of satellite observations associated with the emission of the greenhouse gas in a first region of interest from a plurality of satellite data sources, respectively; fusing the plurality of satellite observations to generate a fused input data set; and using an emission estimation model to generate a first emission estimate of the greenhouse gas in the first region of interest based on the fused input data set.

In some embodiments, the process further includes enhancing the fused input data set to be inputted into the emission estimation model using an industrial gas observation associated with the first region of interest.

In some embodiments, to enhance the fused input data set to be inputted into the emission estimation model using the industrial gas observation, the process further includes applying the industrial gas observation to the emission estimation model to estimate one or more model parameters associated with the emission estimation model.

In some embodiments, to use the emission estimation model to generate the first emission estimate of the greenhouse gas in the first region of interest, the process further includes applying the fused input data set to the emission estimation model to generate the first emission estimate of the greenhouse gas based on the one or more model parameters.

In some embodiments, the emission estimation model includes a Gaussian plume model. The one or more model parameters include an extent of a plume in the Gaussian plume model.

In some embodiments, the process further includes associating the first emission estimate of the greenhouse gas with one or more first observations of one or more reference resources in the first region of interest to generate a training data set. The training data set is stored in an association database.

In some embodiments, the one or more reference resources include at least one of an industrial gas resource, a land surface temperature resource, or a shortwave infrared heat resource. The one or more first observations include at least one of the following that is associated with the first region of interest: an industrial gas observation, a land surface temperature observation, or a shortwave infrared heat observation.

In some embodiments, the process further includes deriving a mapping relationship between the emission of the greenhouse gas and the one or more reference resources based on the association database.

In some embodiments, the process further includes: obtaining one or more second observations of the one or more reference resources in a second region of interest where no satellite observation associated with the emission of the greenhouse gas is available from the plurality of satellite data sources; and determining a second emission estimate of the greenhouse gas in the second region of interest based on the mapping relationship and the one or more second observations of the one or more reference resources.

In some embodiments, the first region of interest includes a geographical region that is divided into a plurality of grids. The first emission estimate of the greenhouse gas in the first region of interest includes a plurality of greenhouse-gas emission values for the plurality of grids, respectively.

In some embodiments, the process further includes: identifying one or more emission sources within a grid in the plurality of grids; and disaggregating a greenhouse-gas emission value associated with the grid to one or more disaggregated greenhouse-gas emission values for the one or more emission sources.

In some embodiments, to disaggregate the greenhouse-gas emission value associated with the grid to the one or more disaggregated greenhouse-gas emission values for the one or more emission sources, the process further includes: for each emission source from the one or more emission sources, obtaining one or more observations of one or more reference resources associated with the grid, respectively; using the one or more observations of the one or more reference resources to determine an initial emission estimate of the greenhouse gas for the emission source based on a mapping relationship between the emission of the greenhouse gas and the one or more reference resources so that one or more initial emission estimates of the greenhouse gas are generated for the one or more emission sources; and disaggregating the greenhouse-gas emission value associated with the grid to the one or more disaggregated greenhouse-gas emission values based on the one or more initial emission estimates of the greenhouse gas for the one or more emission sources.

In some embodiments, the process further includes: identifying, from the one or more emission sources, an emission source having a potentially falsified emission report based on a disaggregated greenhouse-gas emission value associated with the emission source; and providing a notification to prioritize a field inspection on the emission source having the potentially falsified emission report.

In some embodiments, the first region of interest includes a geographical region that is divided into a plurality of grids. To fuse the plurality of satellite observations to generate the fused input data set, the process further includes: resampling each satellite observation into one or more resampled observation values associated with one or more grids in the plurality of grids; and for each grid in the plurality of grids, determining an availability of resampled observation values associated with the grid, and generating a fused input value associated with the grid based on the availability of resampled observation values associated with the grid.

In some embodiments, each satellite observation includes one or more initial observation values associated with the first region of interest. To resample each satellite observation into the one or more resampled observation values associated with the one or more grids, the process further includes applying a geographically-weighted method to generate the one or more resampled observation values associated with the one or more grids based on the one or more initial observation values.

In some embodiments, the greenhouse gas includes CO₂. The plurality of satellite observations include a plurality of observations of column-averaged dry-air mole fractions of CO₂ in the atmosphere (xCO₂). The plurality of xCO₂ satellite observations are associated with the first region of interest and obtained from a plurality of xCO₂ data sources, respectively.

According to yet another aspect of the present disclosure, a non-transitory computer-readable storage medium is disclosed. The computer-readable storage medium is configured to store instructions which, in response to an execution by a processor, cause the processor to perform a process including: receiving a plurality of satellite observations associated with the emission of the greenhouse gas in a first region of interest from a plurality of satellite data sources, respectively; fusing the plurality of satellite observations to generate a fused input data set; and using an emission estimation model to generate a first emission estimate of the greenhouse gas in the first region of interest based on the fused input data set.

The foregoing description of the specific implementations can be readily modified and/or adapted for various applications. Therefore, such adaptations and modifications are intended to be within the meaning and range of equivalents of the disclosed implementations, based on the teaching and guidance presented herein.

The breadth and scope of the present disclosure should not be limited by any of the above-described exemplary implementations, but should be defined only in accordance with the following claims and their equivalents.

What is claimed is:

1. A computer-implemented method for monitoring emission of a greenhouse gas, comprising:
receiving a plurality of satellite observations associated with the emission of the greenhouse gas in a first region of interest from a plurality of satellite data sources, respectively;
fusing the plurality of satellite observations to generate a fused input data set; and
using an emission estimation model to generate a first emission estimate of the greenhouse gas in the first region of interest based on the fused input data set.

2. The method of claim 1, further comprising:
enhancing the fused input data set to be inputted into the emission estimation model using an industrial gas observation associated with the first region of interest.

3. The method of claim 2, wherein enhancing the fused input data set to be inputted into the emission estimation model using the industrial gas observation comprises:
applying the industrial gas observation to the emission estimation model to estimate one or more model parameters associated with the emission estimation model.

4. The method of claim 3, wherein using the emission estimation model to generate the first emission estimate of the greenhouse gas in the first region of interest comprises:
applying the fused input data set to the emission estimation model to generate the first emission estimate of the greenhouse gas based on the one or more model parameters.

5. The method of claim 3, wherein the emission estimation model comprises a Gaussian plume model, and
wherein the one or more model parameters comprise an extent of a plume in the Gaussian plume model.

6. The method of claim 1, further comprising:
associating the first emission estimate of the greenhouse gas with one or more first observations of one or more reference resources in the first region of interest to generate a training data set; and
storing the training data set in an association database.

7. The method of claim 6, wherein the one or more reference resources comprise at least one of an industrial gas resource, a land surface temperature resource, or a shortwave infrared heat resource, and
wherein the one or more first observations comprise at least one of the following that is associated with the first region of interest: an industrial gas observation, a land surface temperature observation, or a shortwave infrared heat observation.

8. The method of claim 6, further comprising:
deriving a mapping relationship between the emission of the greenhouse gas and the one or more reference resources based on the association database.

9. The method of claim 8, further comprising:
obtaining one or more second observations of the one or more reference resources in a second region of interest where no satellite observation associated with the emission of the greenhouse gas is available from the plurality of satellite data sources; and
determining a second emission estimate of the greenhouse gas in the second region of interest based on the mapping relationship and the one or more second observations of the one or more reference resources.

10. The method of claim 1, wherein the first region of interest comprises a geographical region that is divided into a plurality of grids, and the first emission estimate of the greenhouse gas in the first region of interest comprises a plurality of greenhouse-gas emission values for the plurality of grids, respectively.

11. The method of claim 10, further comprising:
identifying one or more emission sources within a grid in the plurality of grids; and
disaggregating a greenhouse-gas emission value associated with the grid to one or more disaggregated greenhouse-gas emission values for the one or more emission sources.

12. The method of claim 11, wherein disaggregating the greenhouse-gas emission value associated with the grid to the one or more disaggregated greenhouse-gas emission values for the one or more emission sources comprises:
for each emission source from the one or more emission sources, obtaining one or more observations of one or more reference resources associated with the grid, respectively;
using the one or more observations of the one or more reference resources to determine an initial emission estimate of the greenhouse gas for the emission source based on a mapping relationship between the emission of the greenhouse gas and the one or more reference resources so that one or more initial emission estimates of the greenhouse gas are generated for the one or more emission sources; and
disaggregating the greenhouse-gas emission value associated with the grid to the one or more disaggregated greenhouse-gas emission values based on the one or more initial emission estimates of the greenhouse gas for the one or more emission sources.

13. The method of claim 11, further comprising:
identifying, from the one or more emission sources, an emission source having a potentially falsified emission report based on a disaggregated greenhouse-gas emission value associated with the emission source; and
providing a notification to prioritize a field inspection on the emission source having the potentially falsified emission report.

14. The method of claim 1, wherein the first region of interest comprises a geographical region that is divided into a plurality of grids, and fusing the plurality of satellite observations to generate the fused input data set comprises:
resampling each satellite observation into one or more resampled observation values associated with one or more grids in the plurality of grids; and
for each grid in the plurality of grids,
determining an availability of resampled observation values associated with the grid; and
generating a fused input value associated with the grid based on the availability of resampled observation values associated with the grid.

15. The method of claim 14, wherein each satellite observation comprises one or more initial observation values associated with the first region of interest, and
resampling each satellite observation into the one or more resampled observation values associated with the one or more grids comprises:
applying a geographically-weighted method to generate the one or more resampled observation values associated with the one or more grids based on the one or more initial observation values.

16. The method of claim 1, wherein the greenhouse gas comprises carbon dioxide (CO₂), and
wherein the plurality of satellite observations comprise a plurality of observations of column-averaged dry-air mole fractions of CO₂ in the atmosphere (xCO₂), the plurality of xCO₂ satellite observations being associated with the first region of interest and obtained from a plurality of xCO₂ data sources, respectively.

17. A system for monitoring emission of a greenhouse gas, comprising:
a memory configured to store instructions; and
a processor coupled to the memory and configured to execute the instructions to perform a process comprising:
receiving a plurality of satellite observations associated with the emission of the greenhouse gas in a first region of interest from a plurality of satellite data sources, respectively;
fusing the plurality of satellite observations to generate a fused input data set; and
using an emission estimation model to generate a first emission estimate of the greenhouse gas in the first region of interest based on the fused input data set.

18. The system of claim 17, wherein the process further comprises:
enhancing the fused input data set to be inputted into the emission estimation model using an industrial gas observation associated with the first region of interest.

19. The system of claim 18, wherein to enhance the fused input data set to be inputted into the emission estimation model using the industrial gas observation, the process further comprises:
applying the industrial gas observation to the emission estimation model to estimate one or more model parameters associated with the emission estimation model.

20. A non-transitory computer-readable storage medium configured to store instructions which, in response to an execution by a processor, cause the processor to perform a process comprising:
receiving a plurality of satellite observations associated with the emission of the greenhouse gas in a first region of interest from a plurality of satellite data sources, respectively;
fusing the plurality of satellite observations to generate a fused input data set; and
using an emission estimation model to generate a first emission estimate of the greenhouse gas in the first region of interest based on the fused input data set.
